(19) United States
(12) Patent Application Publication
(10) Pub. No.: US 2011/0186049 A1
(43) Pub. Date: Aug. 4, 2011

(54) A SYSTEM FOR ANESTHETIZING WHILST ATTUNING THE TEMPERATURE OF MAMMALS AND METHODS THEREOF

(75) Inventor: Uri RAPOPORT, Moshav Ben Shemen (IL)

(73) Assignee: ASPECT MAGNET TECHNOLOGIES LTD., Shoham (IL)

(21) Appl. No.: 13/058,569

(22) PCT Filed: Aug. 12, 2009
(86) PCT No.: PCT/IL2009/000792

§ 371 Date: Mar. 8, 2011

Related U.S. Application Data
(60) Provisional application No. 61/088,011, filed on Aug. 12, 2008.

(51) Int. Cl. A61M 16/01; A61M 16/04

(52) U.S. Cl. 128/203.29; 128/203.12

(57) ABSTRACT

The present invention discloses methods and a system for anaesthetizing mammals in a thermoregulated confined volume. The system comprises at least one chamber confining a volume in size and shape for accommodating at least one mammal; supplying mechanism for supplying at least one anesthetizing vapor to said at least one chamber; temperature controlling mechanism, in communication with said vapor supplying means, adapted to thermoregulate said vapor to temperature T; and a vapor introducing mechanism, coupled to said supplying means and to said at least one chamber, adapted to introduce said thermoregulated vapor to said at least one chamber; wherein the temperature T within said confined volume is regulated by said thermoregulated vapor.

### FIELD OF THE INVENTION

This invention generally relates to a system for anesthetizing whilst attuning the temperature of one or more mammals and a method of using the system.

### BACKGROUND OF THE INVENTION

Anaesthesia of mammals is required for a variety of purposes, some invasive (e.g. surgery, blood or tissue sample extraction), some none-invasive (such as CT or MRI imaging). Anaesthesia may be achieved by injection of drugs or by inhalation of gas or vapor.

Most of the prior art literature discloses inhaled gas that is administered via mask; however such a device prevents adequate exhalation of anaesthesia and carbon dioxide from the lungs, and may require immobilization of the subject. Superfluous gases that leak from the mask edges are inhaled by the handling staff.

Furthermore, the coupling of the mask to the subject requires special location and adjustment of the subject. The mammal is to be placed in a special orientation. This adjustment is a very time consuming step.

Some prior art anaesthesia systems teaches closed systems, which prevent gas leakage and improve gas mixture control. An example of such teaching can be found in US application no. US20040216737 ('737) to Anderson. The application discloses a method and apparatus for anesthetizing one or more laboratory animals. According to '737, the anesthetic component is introduced into a pressurized gas stream for delivery to laboratory animals. The apparatus includes a vacuum-sensitive check valve. The valve permits the delivery of anesthetic gas to the animal only when the animal inhales. Furthermore '737 discloses a thermoregulatory system that transfers heat to animals as they receive anesthesia. The heat is provided on the exterior of the animal supports where animals are positioned during procedures. Such heating requires special and very complicated system.

Another example is EP application no. EP1752111 ('111) to Tsuyuki. The application discloses an apparatus that prevents the leakage of anesthetic gas, and enables in vivo observation of a specimen such as small laboratory animals.

Anaesthesised small mammals tend to suffer from hypothermia, due to their small body habitus, immobilization during prolonged procedures, low environmental temperatures and influence of anaesthetic drugs on thermoregulatory mechanisms.

Some prior art systems use methods of heat conduction, such as electric blankets, warm dressings and other ways of warming animal supports. Such methods warm only the body surfaces touching the device, may cause burns and increase the required chamber size.

Very often after the animal is anaesthesised, medical procedures are performed. Said medical procedure, most often are or none-invasive such as CT or MRI imaging. The worming method which encorporates electric blankets are incompatible with systems such as MRI, and warm dressings have to be constantly replaced.

Thus, there is still a long felt need for a device that enables injection of anaesthesia to mammals by inhalation, with elimination of gas leakage, whilst controlling of the subjects' temperature within a closed system. It is another object to provide an apparatus that enables the compatibility with imaging modalities such as MRI or NMR.

### SUMMARY OF THE INVENTION

In view of the foregoing, it is one object of the present invention to disclose a system for anesthetizing and warming one or more small mammals, comprising mechanism selected in a non-limiting manner from an anaesthesia incubator system, comprising a chamber for small mammals, connected to a system supplying thermoregulated vapor and to a vapor removal system; and controlled by a thermoregulator and vapor control system.

Another object of the present invention is to disclose a method comprising steps selected in a non limiting manner from a group consisting of obtaining an anaesthesia incubator system as defined in the above; placing at least one small mammal in main chamber; Generating and Heating or cooling vapor; introducing vapor into main mammal chamber; Controlling temperature, humidity, anaesthesia and gas concentration in main mammal chamber according to mammal condition; and purging anaesthesia from main mammal chamber.

Another object of the present invention is to disclose an anaesthesia incubator system as defined above, wherein more than one mammal is anaesthesised simultaneously in the same chamber.

Another object of the present invention is to disclose an anaesthesia incubator system as defined above, wherein the anaesthesised subject is a newborn or a premature baby.

Still another object of the present invention is to disclose an anaesthesia incubator system as defined above, wherein the thermoregulated vapor is introduced before the procedure and than disconnected from the chamber.

It is another object of the present invention to provide a system adapted to anaesthetize mammals in a thermoregulated confined volume. The system comprising:
a. at least one chamber (100) confining a volume in size and shape for accommodating at least one mammal;
b. supplying mechanism (120) for supplying at least one anesthetizing vapor to said at least one chamber (100);
c. temperature controlling mechanism (130), in communication with said vapor supplying mechanism (120), adapted to thermoregulated said vapor to temperature T;
d. vapor introducing mechanism (110) coupled to said supplying mechanism (120) and to said at least one chamber (100), adapted to introduce said thermoregulated vapor to said at least one chamber (100);

The temperature T within said confined volume is regulated by said thermoregulated vapor.

It is another object of the present invention to provide the system as defined above, additionally comprising controlling mechanism (150) adapted to control parameters selected from a group consisting of humidity, vapor concentration and any combination thereof in said chamber (100).

It is another object of the present invention to provide the system as defined above, wherein at least a portion of said chamber is disposable.

It is another object of the present invention to provide the system as defined above, wherein said temperature T is higher than about 0 and lower than about 100 degrees, especially 4 degrees C. to about 42 degrees C.

It is another object of the present invention to provide the system as defined above, wherein said vapor is selected from a group consisting of anesthetic gas, water, oxygen or any combination thereof.

It is another object of the present invention to provide the system as defined above, wherein said temperature controlling mechanism (130) is selected from a group consisting of a thermoregulator.

It is another object of the present invention to provide the system as defined above, additionally comprising extracting mechanism (140) adapted to purge said thermoregulated vapor from said chamber (100).

It is another object of the present invention to provide the system as defined above, wherein said system is adapted to simultaneously anaesthesised more than one mammal in the said chamber.

It is another object of the present invention to provide the system as defined above, wherein said system additionally comprising at least one face-mask to be placed on said mammal, adapted to deliver said vapor to said mammal at ambient temperature.

It is another object of the present invention to provide the system as defined above additionally comprising sealing mechanism adapted to seal said system such that no leakage of said vapor is obtained.

It is another object of the present invention to provide the system as defined above, wherein said system additionally comprising at least one imaging device selected from a group consisting of NMR and/or MRI.

It is another object of the present invention to provide a system adapted to simultaneously anaesthetize a plurality of mammals in a thermoregulated confined volume, comprising:
a. a plurality of chambers (100), each of which is confining a volume in size and shape for accommodating at least one mammal;
b. supplying mechanism (120) for supplying at least one vapor to each of said chambers (100);
c. temperature controlling mechanism (130), in communication with said vapor supplying mechanism (120), adapted to thermoregulate said vapor to temperature T;
d. a plurality of vapor introducing mechanism (110) adapted to introduce said thermoregulated vapor to each of said chambers (100); each of said introducing mechanism is coupled to at least one of said chambers (100);

The temperature T within each of said confined volume is regulated by said thermoregulated vapor.

It is another object of the present invention to provide the system as defined above, additionally comprising controlling mechanism (150) adapted to control parameters selected from a group consisting of humidity, vapor concentration and any combination thereof in each of said chambers (100).

It is another object of the present invention to provide the system as defined above, wherein at least a portion of each of said chambers is disposable.

It is another object of the present invention to provide the system as defined above, wherein said temperature T is higher than about 0 and lower than about 100 degrees, especially 4 degrees C. to about 42 degrees C.

It is another object of the present invention to provide the system as defined above, wherein said vapor is selected from a group consisting of anesthetic gas, water, oxygen or any combination thereof.

It is another object of the present invention to provide the system as defined above, wherein said temperature controlling mechanism (130) is selected from a group consisting of a thermoregulator.

It is another object of the present invention to provide the system as defined above, additionally comprising extracting mechanism (140) adapted to purge said thermoregulated vapor from each of said chambers (100).

It is another object of the present invention to provide the system as defined above, wherein said system additionally comprising at least one face-mask to be placed on said mammal, adapted to deliver said vapor to said mammal at ambient temperature.

It is another object of the present invention to provide the system as defined above, wherein said system additionally comprising sealing mechanism adapted to seal said system such that no leakage of said vapor is obtained.

It is another object of the present invention to provide a system adapted to perform a medical and/or biological procedure on mammals in a thermoregulated confined volume, comprising:
a. at least one chamber (100) confining a volume in size and shape for accommodating at least one mammal;
b. supplying mechanism (120) for supplying at least one vapor to said at least one chamber (100);
c. temperature controlling mechanism (130), in communication with said vapor supplying mechanism (120), adapted to thermoregulate said vapor to temperature T according to predetermined biological needs of said mammal;
d. at least one vapor introducing mechanism (110), each of which is coupled to at least one of said chambers; said introducing mechanism are adapted to introduce said thermoregulated vapor to each of said chambers (100);
e. treating mechanism adapted to perform said medical and/or biological procedure on said mammal.

The temperature T within said confined volume is regulated by said thermoregulated vapor.

It is another object of the present invention to provide the system as defined above, additionally comprising controlling mechanism (150) adapted to control parameters selected from a group consisting of humidity, vapor concentration and any combination thereof in said chamber (100).

It is another object of the present invention to provide the system as defined above, wherein at least a portion of said chamber is disposable.

It is another object of the present invention to provide the system as defined above, wherein said temperature T is higher than about 0 and lower than about 100 degrees, especially 4 degrees C. to about 42 degrees C.

It is another object of the present invention to provide the system as defined above, wherein said vapor is selected from a group consisting of anesthetic gas, water, oxygen or any combination thereof.

It is another object of the present invention to provide the system as defined above, wherein said temperature controlling mechanism (130) is selected from a group consisting of a thermoregulator.

It is another object of the present invention to provide the system as defined above, additionally comprising extracting mechanism (140) adapted to purge said thermoregulated vapor from said chamber (100).

It is another object of the present invention to provide the system as defined above, wherein said system is adapted to simultaneously anaesthesised more than one mammal in the said chamber.

It is another object of the present invention to provide the system as defined above, wherein said system additionally comprising at least one face-mask to be placed on said mammal, adapted to deliver said vapor to said mammal at ambient temperature.

It is another object of the present invention to provide the system as defined above, wherein said treating mechanism is at least one imaging device selected from a group consisting of NMR and/or MM.

It is another object of the present invention to provide the system as defined above, wherein said system additionally comprising sealing mechanism adapted to seal said system such that no leakage of said vapor is obtained.

It is another object of the present invention to provide a method for anaesthetizing mammals. The method comprises steps selected inter alia from:
a. obtaining an anaesthesia chamber;
b. accommodating at least one mammal in said chamber;
c. obtaining an anaesthetic gas sources; said anaesthetic gas is to be delivered to each of said chamber;
d. coupling said anaesthetic gas sources to said chamber;
e. thermoregulating said anaesthetic gas to Temperature T; and,
f. introducing said thermoregulated anaesthetic gas into said chamber thereby regulating said temperature T within said chamber.

It is another object of the present invention to provide the method as defined above, additionally comprising step of treating said mammal.

It is another object of the present invention to provide the method as defined above, wherein said step of treating said mammal additionally comprising step of performing any medical and/or biological procedure on said mammal.

It is another object of the present invention to provide the method as defined above, additionally comprising step of attuning said temperature T to be higher than about 0 and lower than about 100 degrees, especially 4 degrees C. to about 42 degrees C.

It is another object of the present invention to provide the method as defined above, additionally comprising step of selecting said anesthetic vapor from a group consisting of Nitrous oxide (N₂O), Halothane, Enflurane, Isoflurane, Sevoflurane, Desflurane and Xenon, water, oxygen or any combination thereof.

It is another object of the present invention to provide the method as defined above, additionally comprising step of ceasing said step of introducing said thermoregulated anaesthetic gas.

It is another object of the present invention to provide the method as defined above, additionally comprising step of controlling parameters selected from a group consisting of humidity, vapor concentration and any combination thereof in said chamber (100).

It is another object of the present invention to provide the method as defined above, additionally comprising step of placing at least one face-mask on said mammal; and delivering said vapor to said mammal at ambient temperature.

It is another object of the present invention to provide the method as defined above, additionally comprising step of sealing said system such that no leakage of said vapor is obtained.

It is another object of the present invention to provide a method for simultaneously anaesthetizing a plurality of mammals. The method comprises steps selected inter alia from:
a. obtaining a plurality of anaesthesia chambers;
b. accommodating at least one mammal in each of said chamber;
c. obtaining an anaesthetic gas source; said anaesthetic gas is to be delivered to each of said chamber;
d. coupling said anaesthetic gas source to said plurality of chambers;
e. thermoregulating said anaesthetic gas to Temperature T; and,
f. introducing said thermoregulated anaesthetic gas into said chamber thereby regulating said temperature T within said chamber.

It is another object of the present invention to provide the method as defined above, additionally comprising step of treating said mammal.

It is another object of the present invention to provide the method as defined above, wherein said step of treating said mammal additionally comprising step of performing any medical and/or biological procedure on said mammal.

It is another object of the present invention to provide the method as defined above, additionally comprising step of attuning said temperature T to be higher than about 0 and lower than about 100 degrees, especially 4 degrees C. to about 42 degrees C.

It is another object of the present invention to provide the method as defined above, additionally comprising step of selecting said vapor from a group consisting of anesthetic gas, water, oxygen or any combination thereof.

It is another object of the present invention to provide the method as defined above, additionally comprising step of ceasing said step of introducing said thermoregulated anaesthetic gas.

It is another object of the present invention to provide the method as defined above, additionally comprising step of controlling parameters selected from a group consisting of humidity, vapor concentration and any combination thereof in said chamber (100).

It is another object of the present invention to provide the method as defined above, additionally comprising step of placing at least one face-mask on said mammal; and delivering said vapor to said mammal at ambient temperature.

It is another object of the present invention to provide the method as defined above, additionally comprising step of sealing said system such that no leakage of said vapor is obtained.

It is another object of the present invention to provide a method for simultaneously anaesthetizing a plurality of mammals. The method comprises steps selected inter alia from:
a. obtaining a plurality of anaesthesia chambers;
b. accommodating at least one mammal in each of said chamber;
c. obtaining a plurality of anaesthetic gas sources; said anaesthetic gas is to be delivered to each of said chamber;
d. coupling each of said anaesthetic gas sources to at least one of said chambers;
e. thermoregulating said anaesthetic gas to Temperature T; and,
f. introducing said thermoregulated anaesthetic gas into said chamber thereby regulating said temperature T within said chamber.

It is another object of the present invention to provide the method as defined above, additionally comprising step of treating said mammal.

It is another object of the present invention to provide the method as defined above, wherein said step of treating said mammal additionally comprising step of performing any medical and/or biological procedure on said mammal.

It is another object of the present invention to provide the method as defined above, additionally comprising step of attuning said temperature T to be higher than about 0 and lower than about 100 degrees, especially 4 degrees C. to about 42 degrees C.

It is another object of the present invention to provide the method as defined above, additionally comprising step of selecting said vapor from a group consisting of anesthetic gas, water, oxygen or any combination thereof.

It is another object of the present invention to provide the method as defined above, additionally comprising step of ceasing said step of introducing said thermoregulated anaesthetic gas.

It is another object of the present invention to provide the method as defined above, additionally comprising step of controlling parameters selected from a group consisting of humidity, vapor concentration and any combination thereof in said chamber (100).

It is another object of the present invention to provide the method as defined above, additionally comprising step of placing at least one face-mask on said mammal; and delivering said vapor to said mammal at ambient temperature.

It is another object of the present invention to provide the method as defined above, additionally comprising step of sealing said system such that no leakage of said vapor is obtained.

It is another object of the present invention to provide a method for performing at least one medical and/or biological procedure on at least one mammal. The method comprises steps selected inter alia from:
a. obtaining at least one anaesthesia chamber;
b. accommodating at least one mammal in said chamber;
c. obtaining at least one vapor source; said at least one vapor is to be delivered to each of said chamber;
d. coupling at least one of said at least one vapor sources to at least one of said chambers;
e. thermoregulating said at least one vapor to Temperature T;
f. introducing said thermoregulated anaesthetic gas into said chamber thereby regulating said temperature T within said chamber; and,
g. performing said medical and/or biological procedure on said at least one mammal.

It is another object of the present invention to provide the, method as defined above, additionally comprising step of attuning said temperature T to be higher than about 0 and lower than about 100 degrees, especially 4 degrees C. to about 42 degrees C.

It is another object of the present invention to provide the method as defined above, additionally comprising step of selecting said vapor from a group consisting of anesthetic gas, water, oxygen or any combination thereof.

It is another object of the present invention to provide the method as defined above, comprising step of ceasing said step of introducing said thermoregulated anaesthetic gas.

It is another object of the present invention to provide the method as defined above, additionally comprising step of controlling parameters selected from a group consisting of humidity, vapor concentration and any combination thereof in said chamber (100).

It is another object of the present invention to provide the method as defined above, additionally comprising step of placing at least one face-mask on said mammal; and delivering said vapor to said mammal at ambient temperature.

It is another object of the present invention to provide the method as defined above, additionally comprising step of performing another medical procedure on said mammal.

It is another object of the present invention to provide the method as defined above, additionally comprising step of sealing said system such that no leakage of said vapor is obtained.

It is yet another object of the present invention to provide a system adapted to perform a medical and/or biological procedure on mammals in a thermoregulated confined volume. The system comprises inter alia the following:
a. at least one chamber (100) confining a volume in size and shape for accommodating at least one mammal;
b. at least one pretreatment mechanism adapted to accommodate said chamber and to enable pretreatment of said mammal;
c. supplying mechanism (120) for supplying at least one vapor to said at least one chamber (100);
d. temperature controlling mechanism (130), in communication with said supplying mechanism (120), adapted thermoregulate said vapor to temperature T;
e. at least one introducing mechanism (110), each of which is coupled to at least one of said chambers; each of said introducing mechanism is adapted to introduce said thermoregulated vapor to each of said chambers (100);
f. treating mechanism adapted to perform said medical and/or biological procedure on said mammal.

It is another object of the present invention to provide the system as defined above, wherein said pretreatment mechanism are selected form a group consisting of glove box.

It is another object of the present invention to provide the system as defined above, wherein said chamber has a backing support upon which said mammal lays; and a top cover adapted to close and/or seal said chamber.

It is another object of the present invention to provide the system as defined above, additionally comprising controlling mechanism (150) adapted to control parameters selected from a group consisting of humidity, vapor concentration and any combination thereof in said chamber (100).

It is another object of the present invention to provide the system as defined above, wherein at least a portion of said chamber is disposable.

It is another object of the present invention to provide the system as defined above, wherein said temperature T is higher than about 0 and lower than about 100 degrees, especially 4 degrees C. to about 42 degrees C.

It is another object of the present invention to provide the system as defined above, wherein said vapor is selected from a group consisting of anesthetic gas selected from a group consisting of Nitrous oxide (N₂O), Halothane, Enflurane, Isoflurane, Sevoflurane, Desflurane and Xenon, water, oxygen or any combination thereof.

It is another object of the present invention to provide the system as defined above, additionally comprising selection mechanism coupled to said supplying mechanism, introducing mechanism and said chamber for selecting and/or altering between said vapor introduced into said chamber.

It is another object of the present invention to provide the system as defined above, wherein said temperature controlling mechanism (130) is selected from a group consisting of a thermoregulator.

It is another object of the present invention to provide the system as defined above, additionally comprising extracting mechanism (140) adapted to purge said thermoregulated vapor from said chamber (100).

It is another object of the present invention to provide the system as defined above, wherein said system additionally comprising at least one face-mask to be placed on said mammal, adapted to deliver said vapor to said mammal at ambient temperature.

It is another object of the present invention to provide the system as defined above, wherein said treating mechanism is at least one imaging device selected from a group consisting of NMR, MRI, CT, X-ray, ultrasound device, fluorescence device, thermographic device or any combination thereof.

It is lastly an object of the present invention to provide the system as defined above, wherein said system additionally comprising sealing mechanism adapted to seal said system such that no leakage of said vapor is obtained.

### BRIEF DESCRIPTION OF FIGURES

In order to understand the invention and to see how it may be implemented in practice, a few preferred embodiments will now be described, by way of non-limiting example only, with reference to the accompanying drawings, in which:

FIGS. 1-7 schematically presents in an out-of-scale manner an incubator system according to different embodiments of the present invention;

FIGS. 8-12 discloses a method for employing said incubator system.

FIGS. 13a-13d illustrate another embodiment of the present invention.

### DETAILED DESCRIPTION OF THE DRAWINGS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide mechanism and method of anesthetizing and warming mammals.

The term “vapor” refers hereinafter to a mixture of substances suspended in the air, selected in a non-limiting manner from water, oxygen, substances used for induction of anaesthesia, or other components, which can be used for respiration, heating, cooling or humidifying.

The tau “anaesthesia” refers hereinafter to currently inhaled general anesthetic agent selected from a group consisting of Nitrous oxide (N₂O), Halothane, Enflurane, Isoflurane, Sevoflurane, Desflurane and Xenon (which is rarely used) or any combination thereof.

The term “Halothane” refers hereinafter to the IUPAC name 2-bromo-2-chloro-1,1,1-trifluoroethane.

The term “Enflurane” refers hereinafter to the IUPAC name 2-chloro-1-(difluoromethoxy)-1,1,2-trifluoro-ethane.

The term “Isoflurane” refers hereinafter to the IUPAC name 2-chloro-2-(difluoromethoxy)-1,1,1-trifluoro-ethane.

The term “Sevoflurane” refers hereinafter to the IUPAC name 1,1,1,3,3,3-hexafluoro-2-(fluoromethoxy)propane.

The term “Desflurane” refers hereinafter to the IUPAC name 2-(difluoromethoxy)-1,1,1,2-tetrafluoro-ethane.

The term “about” refers hereinafter to a range of 25% below or above the referred value.

The term “incubator” refers hereinafter in a non-limiting manner to an apparatus in which environmental conditions, such as temperature, concentration of gases and humidity, can be controlled.

The term “treating” refers hereinafter in a non-limiting manner to any medical and/or biological procedure to be performed on a mammal. The medical and/or biological procedure can be invasive (e.g. surgery, blood or tissue sample extraction et cetera), or none-invasive (such as CT, MRI imaging, X-ray, ultrasound, fluorescence, thermography, et cetera).

The term “fluorescence” refers hereinafter in a non-limiting manner to luminescence in which the molecular absorption of a photon triggers the emission of another photon with a longer wavelength.

The term “fluorescence” refers hereinafter in a non-limiting manner to an infrared imaging science. Thermographic cameras detect radiation in the infrared range of the electromagnetic spectrum (roughly 900-14,000 nanometers or 0.9-14 μm) and produce images of that radiation.

The term “thermoregulator” refers hereinafter in a non-limiting manner to any device that enables temperature regulation.

The term “glove box” or “glovebox” refers hereinafter in a non-limiting manner to a sealed container that is designed to allow one to manipulate objects while being in a different atmosphere from the object. Usually on the sides of the glove box are two gloves arranged in such a way that one can place his or her hands into the gloves and be able to perform tasks inside the box without breaking the seal or allowing potential injury to the worker. Usually at least a part or all of the glove box's walls are transparent to allow one to see what is being manipulated.

The term “pretreatment” refers hereinafter in a non-limiting manner to any treatment needed to be performed on a mammal prior to performing a medical and/or biological procedure.

The term “temperature controlling mechanism” refers hereinafter in a non-limiting manner to any mechanism enabling thermoregulating (i.e., heating or cooling) of a substrate.

The present invention provides an incubator system adapted to anaesthetize mammals in a thermoregulated confined volume. The system comprises inter alia the following:
a. A chamber (100) confining a volume in size and shape for accommodating at least one mammal.
b. Supplying mechanism (120) for supplying at least one vapor to each of the chambers (100).
c. Temperature controlling mechanism (130), in communication with the supplying mechanism (120), adapted to thermoregulate the vapor to temperature T.
d. Introducing mechanism (110) adapted to introduce the thermoregulated vapor to the chamber (100).

The temperature T within said confined volume is regulated by said thermoregulated vapor.

The present invention also provides an incubator system for simultaneously anaesthetize a plurality of mammals in a thermoregulated confined volume. The system comprises:
a. A plurality of chambers (100), each of which is confining a volume in size and shape for accommodating at least one mammal.
b. Supplying mechanism (120) for supplying at least one vapor to each of the chambers (100).
c. Temperature controlling mechanism (130), in communication with the supplying mechanism (120), adapted to thermoregulate the vapor to temperature T (i.e., attune the temperature T of the thermoregulated vapor).
d. introducing mechanism (110) adapted to introduce the thermoregulated vapor to each of the chambers (100);

The temperature T within said confined volume is regulated by said thermoregulated vapor.

The present invention also provides a method for anaesthetizing mammals. The method comprises steps selected inter alia from:
a. obtaining an anaesthesia chamber.
b. accommodating at least one mammal in the chamber.
c. Obtaining an anaesthetic gas sources. The anaesthetic gas is to be delivered to each of said chamber.
d. Coupling the anaesthetic gas sources to said chamber.
e. thermoregulating the anaesthetic gas to Temperature T.
f. introducing the thermoregulated anaesthetic gas into the chamber whilst simultaneously regulating the temperature T of said anaesthetic gas.
g. treating the mammal.

The temperature T within said confined volume is regulated by said thermoregulated vapor. The present invention also provides a method for simultaneously anaesthetizing a plurality of mammals. The method comprises steps selected inter alia from:
a. Obtaining a plurality of anaesthesia chambers.
b. Accommodating at least one mammal in each of the chamber.
c. Obtaining an anaesthetic gas source. The anaesthetic gas is to be delivered to each of said chamber.
d. Coupling the anaesthetic gas source to the plurality of chambers.
e. thermoregulating said anaesthetic gas to Temperature T.
f. introducing said thermoregulated anaesthetic gas into each of said chambers whilst simultaneously regulating said temperature T of said anaesthetic gas.

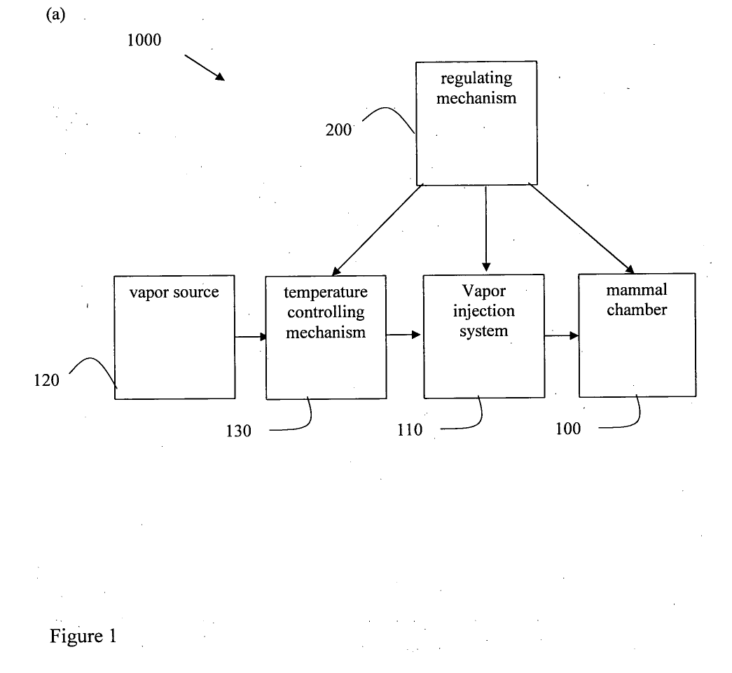

Reference is now made to FIG. 1 which illustrates one embodiment of the present invention. According to that embodiment, an incubator system (1000) for anaesthetizing mammals in a thermoregulated confined volume is disclosed. The system comprises, in a non-limiting manner, the following a chamber for housing at least one mammal (100). The chamber is confining a volume in size and shape for accommodating at least one mammal. The system also comprises at least one vapor source 120 for supplying vapor to the chamber 100. The vapor source is coupled to a temperature controlling system 130 for thermoregulating (i.e., heating or cooling) the vapor to temperature T. One the vapors are thermoregulated (heated or cooled) it is introduced into the chamber 100 via the infusion (introducing) system 110.

The temperature T is regulated to be any desired temperature. The temperature, T can be higher than about 0 and lower than about 100 degrees, especially 4 degrees C. to about 42 degrees C.

The temperature controlling system i.e., the thermoregulatory system or the thermoregulator 130 are provided to achieve and maintain a set temperature within the chamber that accumulates. By controlling the temperature of the animal supports, the body temperature of the laboratory animal may be maintained within a desired range to ensure the well-being of the animal and facilitate the successful completion of the procedure.

The thermoregulated anaesthetic vapor encircles the chamber and hence the mammal and warms it as in an incubator, without the need for a warming pad or an electric blanket. The heat or cold convects around the mammal, and no burns are expected. The mammals' temperature can be monitored, and the vapor temperature can be regulated accordingly.

It should be emphasized that the fact that the anaesthetic vapor is introduce into the chamber 100 mechanism that no mask is required to anaesthetize the mammal. Therefore, no special arrangement and preparation of the mammal is needed. The mammal can be place in any orientation within the chamber.

The system as defined above additionally comprises sealing elements adapted to seal said system such that no gas will leak out of the chamber. Said sealing mechanism ensures that the handling staff will not inhale the gas.

According to a preferred embodiment, the vapor is selected from a group consisting of anesthetic gas e.g., Nitrous oxide (N₂O), Halothane, Enflurane, Isoflurane, Sevoflurane, Desflurane and Xenon, water, oxygen or any combination thereof.

The system as defined above may additionally or alternatively comprise selector mechanism for selecting or altering between the different vapors.

According to another preferred embodiment at least a portion of the chamber is disposable.

According to another preferred embodiment, the system 1000 further comprises at least one face-mask to be placed on the mammal's face. The face-mask is adapted to deliver a non-thermoregulated (heated or cooled) vapor to the mammal (i.e., at ambient temperature).

According to another preferred embodiment, the system 1000 further comprises sealing mechanism adapted to seal the system such that no leakage of the vapor is obtained outside the system.

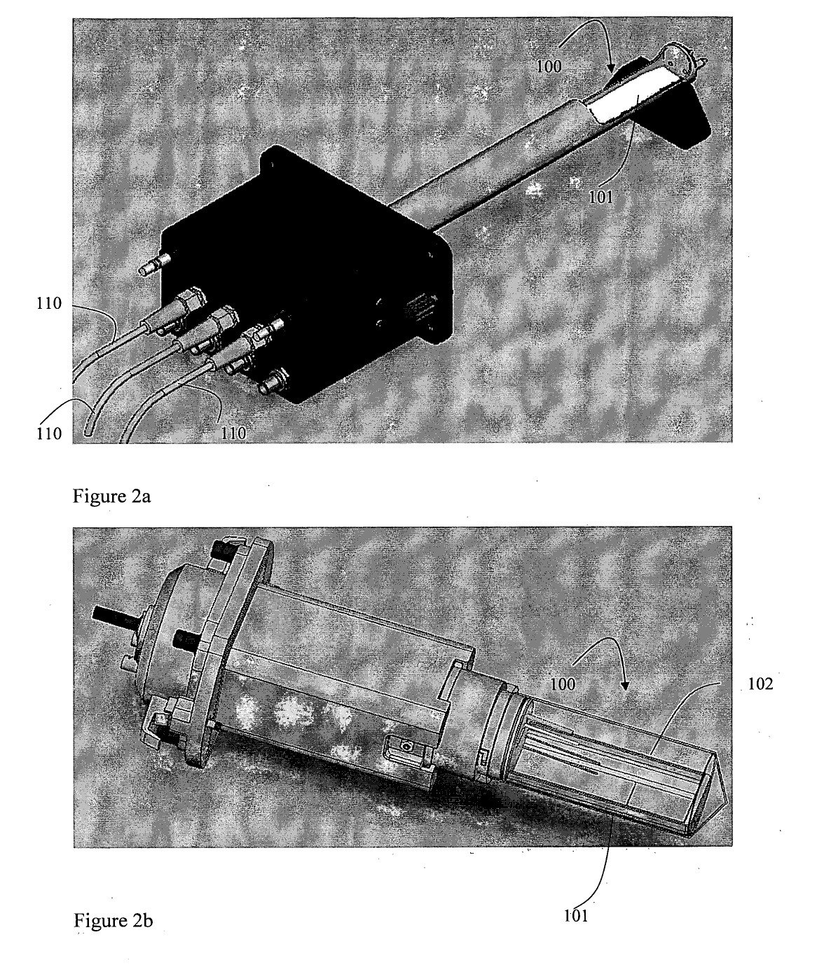

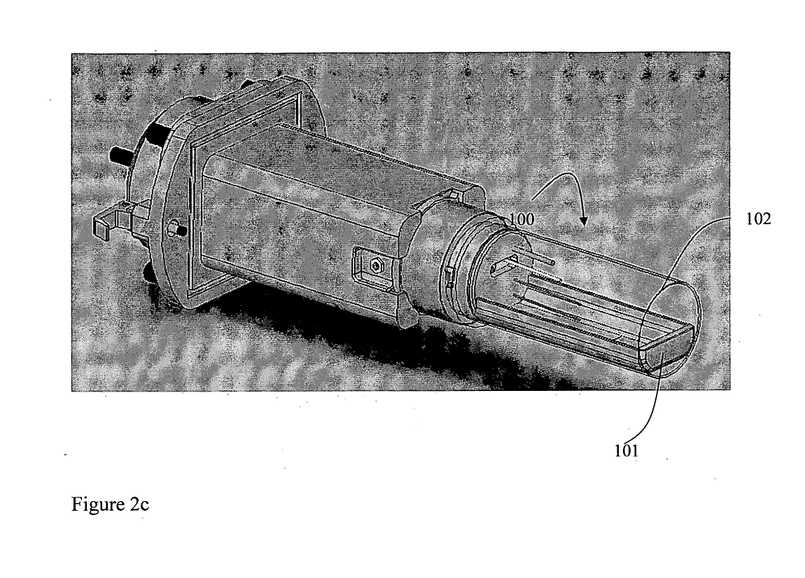

Reference is now made to FIGS. 2a-2c illustrating said incubator system (1000).

According to one specific embodiment, the chamber 100 has a backing support (i.e., a bed) 101 upon which the mammal is laid; and a top cover 102 adapted to close and seal the chamber.

The chamber is coupled to the introducing (infusion) mechanism 110 which are adapted to supply at least one thermoregulated vapor into the chamber.

According to one embodiment of the present invention the top cover and/or the backing support is disposable and adapted for a single use.

It is emphasized that the system is enabled to provide the mammal within the chamber 100 one kind of thermoregulated vapor or several kinds simultaneously or in a batch like manner.

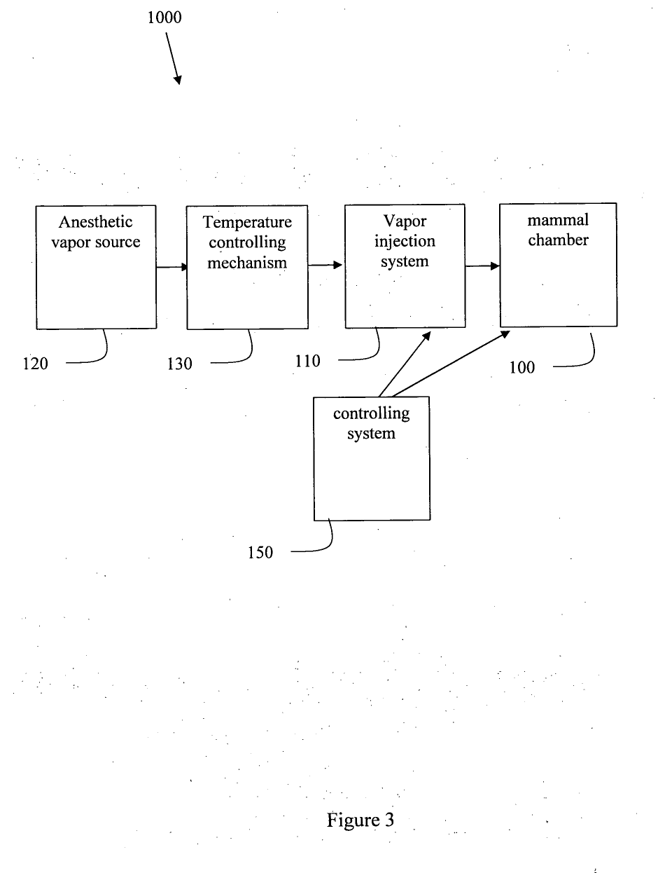

Reference is now made to FIG. 3, schematically illustrating another embodiment (1000) of the present invention. According to that embodiment the chamber 100 and the infusion system 110 are coupled to controlling system 150 for controlling and regulating the humidity, vapor concentration and any combination thereof in the chamber 100.

As described above, the vapor may be selected from a group consisting of anesthetic gas, e.g., Nitrous oxide (N₂O), Halothane, Enflurane, Isoflurane, Sevoflurane, Desflurane and Xenon, water, oxygen or any combination thereof.

Also as described above, the system may additionally or alternatively comprise selection mechanism adapted for selecting or altering between the different vapors introduced into the chamber.

Yet again the system 100 may comprise extracting mechanism (140) to purge said thermoregulated vapor from the chamber (100).

According to another preferred embodiment, the system according to each embodiment described above, wherein at least a portion of the chamber is disposable.

According to another preferred embodiment, the system according to each embodiment described above, the system 1000 further comprises at least one face-mask to be placed on the mammal's face. The face-mask is adapted to deliver a non-heated (or non-cooled) vapor to the mammal (i.e., at ambient temperature).

According to another preferred embodiment, the system according to each embodiment described above, the system 1000 further comprises sealing mechanism adapted to seal the system such that no leakage of the vapor is obtained outside the system.

As mentioned above, according to one specific embodiment, the chamber 100 has a backing support (i.e., a bed) 101 upon which the mammal is laid; and a top cover 102 adapted to close and seal the chamber. According to another embodiment, the top cover and/or the backing support are disposable and are adapted for a single use.

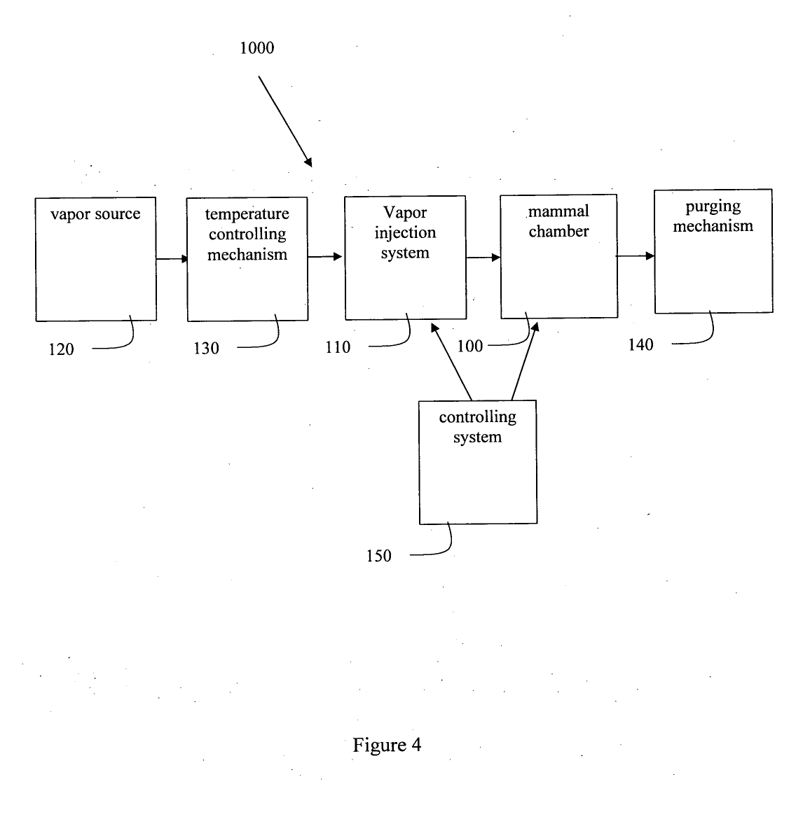

Reference is now made to FIG. 4, schematically illustrating another embodiment of the invention. The incubator system (1000) as described in any of the embodiments described above additionally comprises an extracting mechanism 140 for purging the vapor from the chamber 100.

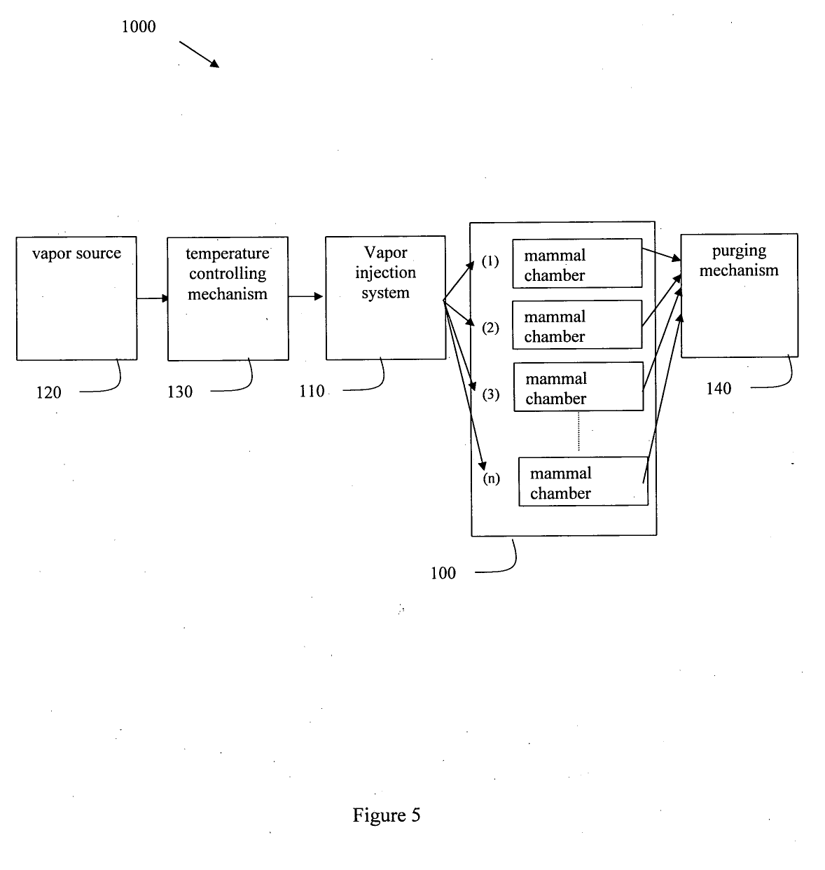

Reference is now made to FIG. 5, schematically illustrating another embodiment of the invention. The incubator system (1000) as described in any of the embodiments described above, in which the system comprises n chambers. N is an integer greater than 1. Each of said n chambers is adapted to accommodate at least one mammal. The incubator system (1000) is hence adapted for anaesthesised a plurality of mammals simultaneously. Each of the chambers could be in different size for accommodating different mammals.

As mentioned above, according to one specific embodiment, each of the chambers 100 has a backing support (i.e., a bed) 101 upon which the mammal is laid; and a top cover 102 adapted to close and seal the chamber. According to another embodiment, the top cover and/or the backing support are disposable and are adapted for a single use.

According to another embodiment of the invention, an incubator system (1000) according to any of the embodiments described above, wherein the anaesthesised subject (i.e. the mammal) is selected from a group consisting of a newborn or a premature baby.

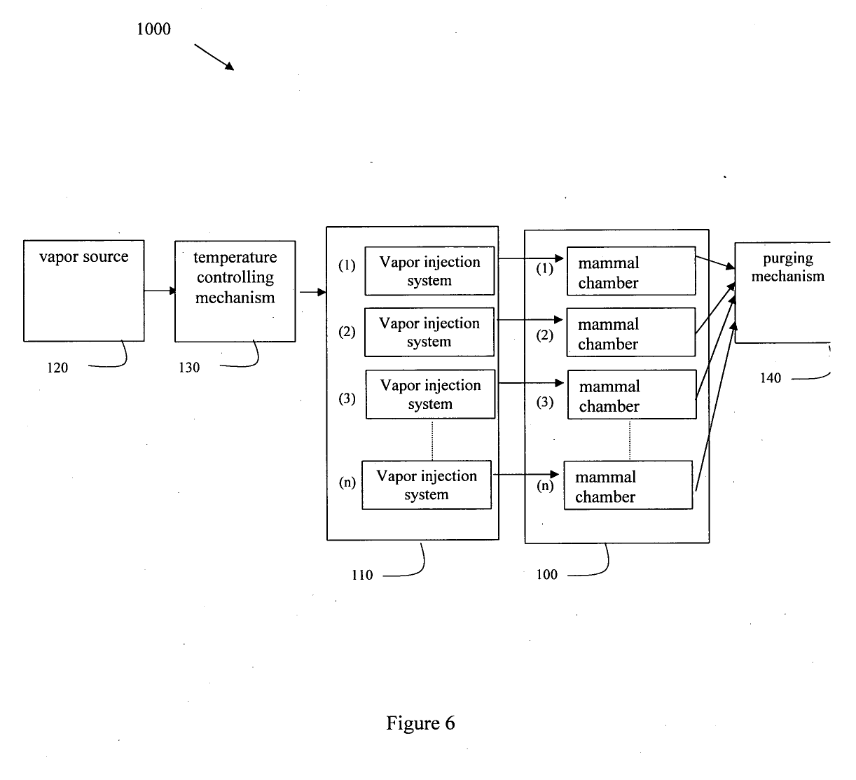

Reference is now made to FIG. 6, which illustrate to another embodiment of the present invention. According to that embodiment the incubator system (1000) comprises a plurality of infusion systems (110). Each infusion system (110) is coupled to at least one chamber 100. Said embodiment is especially needed when there a need to provide various dosages of the vapor (i.e., different concentration) to each chamber. This embodiment may be used when different animals are accommodated in said plurality of chambers such that each mammal should be administered with different dosages of said vapor.

As mentioned above, according to one specific embodiment, the chamber 100 has a backing support (i.e., a bed) 101 upon which the mammal is laid; and a top cover 102 adapted to close and/or seal the chamber. According to another embodiment, the top cover and/or the backing support are disposable and are adapted for a single use.

As described above, according to one embodiment of the present invention the system may additionally comprise controlling, mechanism (150) for controlling parameters selected from a group consisting of humidity, vapor concentration and any combination thereof in the chamber (100).

Furthermore, as described above, according to one embodiment of the present invention at least a portion of said chamber is disposable.

Yet again, as described above, according to one embodiment of the present invention, the regulating mechanism 130 are adapted to attune the temperature of the thermoregulated vapor to be any desired temperature and more specifically to be higher than about 0 and lower than about 100 degrees, especially 4 degrees C. to about 42 degrees C.

As mentioned above, the vapor is selected from a group consisting of anesthetic gas e.g., Nitrous oxide (N₂O), Halothane, Enflurane, Isoflurane, Sevoflurane, Desflurane and Xenon, water, oxygen or any combination thereof.

Furthermore, the system may additionally or alternatively comprise selection mechanism adapted for selecting or altering between the different vapors introduced into the chamber.

Moreover, the system may additionally comprise extracting mechanism (140) for purging the thermoregulated vapor from said chamber (100). Thus, preventing the accumulation of said vapor in said chamber.

Again, as described above the system may additionally comprise at least one face-mask to be placed on said mammal. The face-mask is adapted to deliver said vapor to said mammal at ambient temperature.

Yet again, as described above the system may additionally comprising sealing mechanism adapted to seal said system such that no leakage of said vapor is obtained.

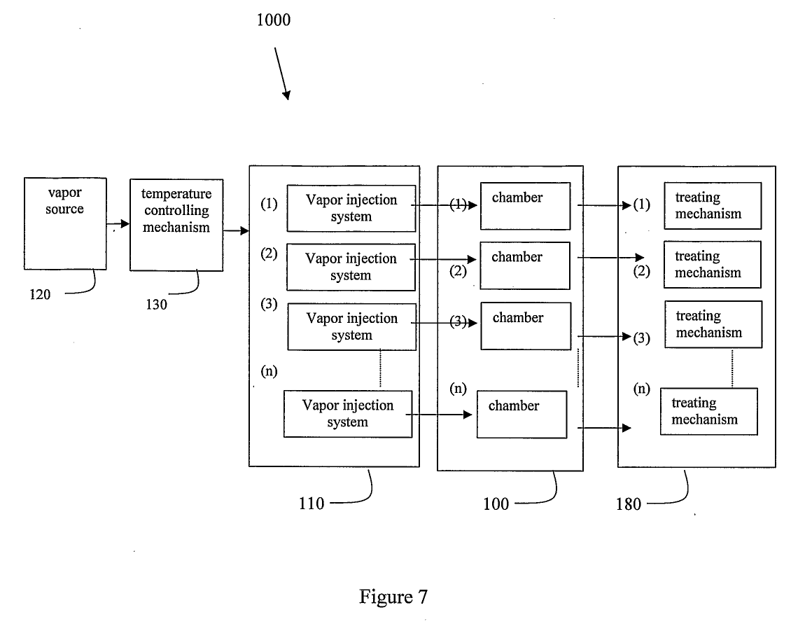

Reference is now made to FIG. 7, which illustrates another embodiment of the present invention. According to that embodiment, the system 1000 comprises at least one chamber (100) to accommodate at least one mammal, supplying mechanism (120) for supplying at least one vapor to at least one chamber (100), temperature controlling system (130), in communication with the supplying mechanism (120), for thermoregulating the vapor to temperature T. the system also comprises at least one introducing (infusion) mechanism (110), each of which is coupled to at least one of the chambers (100). The introducing (infusion) mechanism are adapted to introduce the thermoregulated vapor to each of the chambers (100). The regulating mechanism (130) are adapted to attune the temperature T of the thermoregulated vapor.

The system may further be in communication with treating mechanism 180 adapted to perform any medical and/or biological procedure on the mammal accommodated within the chambers.

As mentioned above, according to one specific embodiment, each of the chambers 100 has a backing support (i.e., a bed) 101 upon which the mammal is laid; and a top cover 102 adapted to close and/or seal the chamber. According to another embodiment, the top cover and/or the backing support are disposable and are adapted for a single use.

According to one embodiment of the present invention, the vapor is selected from a group consisting of anesthetic gas e.g., Nitrous oxide (N₂O), Halothane, Enflurane, Isoflurane, Sevoflurane, Desflurane and Xenon, water, oxygen or any combination thereof.

Also as described above, the system may additionally or alternatively comprise selection mechanism adapted for selecting or altering between the different vapors introduced into the chamber.

According to another embodiment of the present invention, the system as describe above additionally comprises controlling mechanism (150) adapted to control parameters selected from a group consisting of humidity, vapor concentration and any combination thereof in said chamber (100).

According to another embodiment of the present invention at least a portion of said chamber is disposable.

According to another embodiment of the present invention, the regulating mechanism (130) are adapted to attune the temperature T of the thermoregulated vapor to be any desired temperature and especially, higher than about 0 and lower than about 100 degrees, especially 4 degrees C. to about 42 degrees C.

According to another embodiment of the present invention the system additionally comprises extracting mechanism (140) adapted to purge said thermoregulated vapor from said chamber (100).

According to another embodiment of the present invention the system additionally comprises at least one face-mask to be placed on said mammal, adapted to deliver said vapor to said mammal at ambient temperature.

According to another embodiment of the present invention the system additionally comprises sealing mechanism adapted to seal said system such that no leakage of said vapor is obtained.

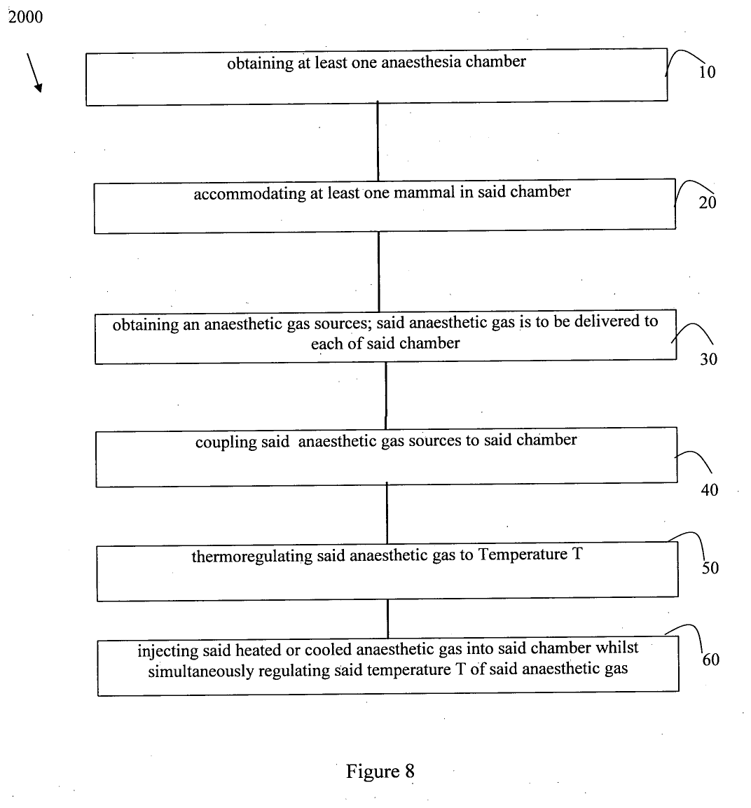

Another object of the present invention is to disclose a method (2000) for anaesthetizing mammals. Reference is now made to FIG. 8, schematically illustrating said method. The method comprises steps selected in a non limiting manner from:
a. Obtaining an anaesthesia chamber (10).
b. Accommodating at least one mammal in the chamber (20).
c. Obtaining an anaesthetic gas sources. The anaesthetic gas is to be delivered to each of the chamber (30).
d. Coupling the anaesthetic gas sources to the chamber (40)
e. thermoregulating the anaesthetic gas to Temperature T (50); and,
f. Introducing the thermoregulated anaesthetic gas into the chamber whilst simultaneously regulating the temperature T of the anaesthetic gas (60).

According to another embodiment of the present invention, the method additionally comprising the step of treating the mammal. It is emphasized that the step of treating the mammal additionally refers to performing any medical and/or biological procedure on said mammal. The medical procedure can be invasive (e.g. surgery, blood or tissue sample extraction et cetera), or none-invasive (such as CT or MRI imaging, X-ray, ultrasound, fluorescence, thermography, et cetera).

According to another embodiment of the present invention, the method additionally comprising the step of entirely filing the chamber with the vapor.

According to another embodiment of the present invention, the method additionally comprising the step of at least partially purging the chamber from the vapor.

According to another embodiment of the present invention the method as defined above, additionally comprising step of attuning the temperature T to be any desired temperature. More specifically said step of attuning the temperature T is for temperature higher than about 0 and lower than about 100 degrees, especially 4 degrees C. to about 42 degrees C.

According to another embodiment of the present invention the method as defined above, additionally comprising step of selecting said vapor from a group consisting of anesthetic gas e.g., Nitrous oxide (N₂O), Halothane, Enflurane, Isoflurane, Sevoflurane, Desflurane and Xenon, water, oxygen or any combination thereof.

According to another embodiment of the present invention, the method additionally comprising the step of altering the vapor introduced into the chamber.

According to another embodiment of the present invention the method as defined above, additionally comprising step of extracting said anaesthesia from each of said chambers.

According to another embodiment of the present invention the method as defined above, additionally comprising step of ceasing the step of introducing said thermoregulated anaesthetic gas.

According to another embodiment of the present invention, the method additionally comprises step of controlling the temperature, humidity, anaesthesia and gas concentration in the chamber according to mammal condition.

According to another embodiment of the present invention, the method additionally comprises step of ceasing the infusion of said thermoregulated anaesthetic gas into said chamber.

According to another embodiment of the present invention, the method additionally comprises step of placing at least one face-mask on said mammal; and delivering said vapor to said mammal at ambient temperature.

According to another embodiment of the present invention, the method additionally comprises step of sealing said system such that no leakage of said vapor is obtained.

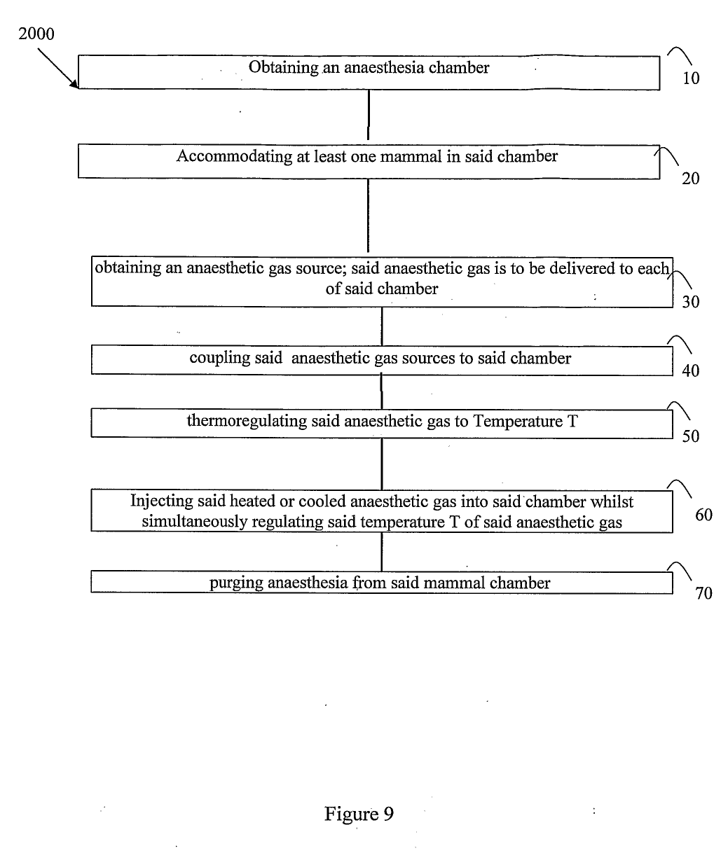

According to another embodiment of the present invention, the method (2000) additionally comprises step of purging anaesthesia from the mammal chamber (see FIG. 9).

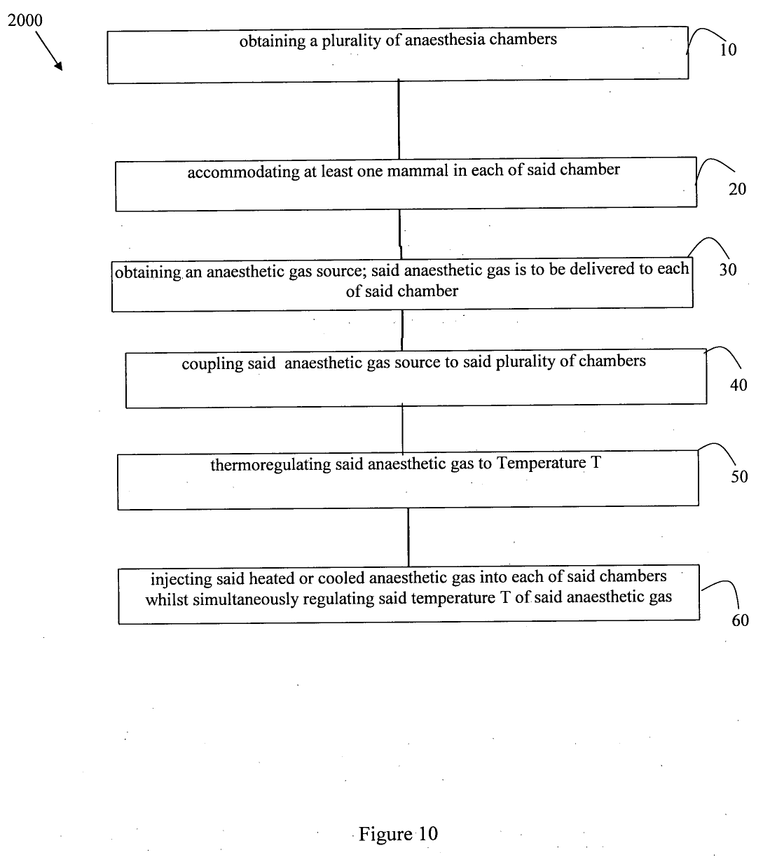

Reference is now made to FIG. 10, disclosing another possible method (2000) for anesthetizing and warming simultaneously anaesthetizing a plurality of mammals. Firstly a plurality of anaesthesia chambers is obtained (10). Next, at least one mammal is accommodated in each of the chamber (20). The next step is to obtain an anaesthetic gas source (30). Next, the anaesthetic gas source is coupled to the plurality of chambers (40). Next, the anaesthetic gas is thermoregulated to Temperature T (50). Finally, the thermoregulated anaesthetic gas is introduced into each of the chambers whilst simultaneously regulating the temperature T of the anaesthetic gas (60).

According to another embodiment of the present invention, the method additionally comprises step of treating the mammal. It is acknowledged that treating the mammal refers to performing any medical and/or biological procedure on said mammal. The medical and/or biological procedure can be invasive (e.g. surgery, blood or tissue sample extraction et cetera), or none-invasive (such as CT or MRI imaging, X-ray, ultrasound, fluorescence, thermography, et cetera).

According to another embodiment of the present invention the method as defined above, additionally comprising step of attuning the temperature T to be any desired temperature. More specifically said step of attuning the temperature T is for temperature higher than about 0 and lower than about 100 degrees, especially 4 degrees C. to about 42 degrees C.

According to another embodiment of the present invention, the method additionally comprising the step of entirely filing the chamber with the vapor.

According to another embodiment of the present invention, the method additionally comprising the step of at least partially purging the chamber from the vapor.

According to another embodiment of the present invention the method as defined above, additionally comprising step of selecting said vapor from a group consisting of anesthetic gas e.g., Nitrous oxide (N₂O), Halothane, Enflurane, Isoflurane, Sevoflurane, Desflurane and Xenon, water, oxygen or any combination thereof.

According to another embodiment of the present invention, the method additionally comprising the step of altering the vapor introduced into the chamber.

According to another embodiment of the present invention the method as defined above, additionally comprising step of extracting said anaesthesia from each of said chambers.

According to another embodiment of the present invention the method as defined above, additionally comprising step of ceasing the step of introducing said thermoregulated anaesthetic gas.

According to another embodiment of the present invention the method as defined above, additionally comprising step of controlling parameters selected from a group consisting of humidity, vapor concentration and any combination thereof in said chamber.

According to another embodiment of the present invention, the method additionally comprises step of ceasing the infusion of said thermoregulated anaesthetic gas into said chamber.

According to another embodiment of the present invention, the method additionally comprises step of placing at least one face-mask on said mammal; and delivering said vapor to said mammal at ambient temperature.

According to another embodiment of the present invention, the method additionally comprises step of sealing said system such that no leakage of said vapor is obtained.

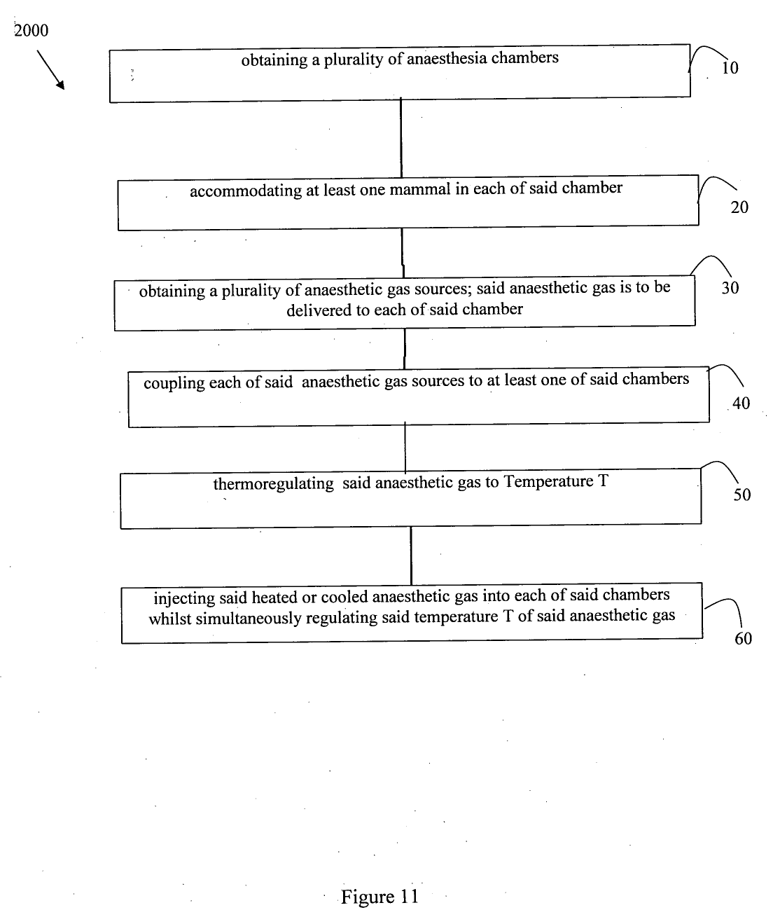

Reference is now made to FIG. 11, disclosing another possible method (2000) for anesthetizing and warming simultaneously anaesthetizing a plurality of mammals. Firstly plurality of anaesthesia chambers are obtained (10). Next, at least one mammal is accommodated in each of the chamber (20). The next step is to obtain a plurality of anaesthetic gas sources (30). Next, each of the anaesthetic gas source is coupled to at least one of the chambers (40). Next, the anaesthetic gas is thermoregulated to Temperature T (50). Finally, the thermoregulated anaesthetic gas is introduced into each of the chambers whilst simultaneously regulating the temperature T of the anaesthetic gas (60).

According to one embodiment of the present invention the method as defined above, additionally comprising a step of treating the mammal, in which the step of treating the mammal additionally comprising step of performing any medical and/or biological procedure on said mammal. The medical and/or biological procedure can be invasive (e.g. surgery, blood or tissue sample extraction et cetera), or none-invasive (such as CT or MRI imaging, X-ray, ultrasound, fluorescence, thermography, et cetera).

According to another embodiment of the present invention the method as defined above, additionally comprising step of attuning the temperature T to be any desired temperature. More specifically said step of attuning the temperature T is for temperature higher than about 0 and lower than about 100 degrees, especially 4 degrees C. to about 42 degrees C.

According to another embodiment of the present invention, the method additionally comprising the step of entirely filing the chamber with the vapor.

According to another embodiment of the present invention, the method additionally comprising the step of at least partially purging the chamber from the vapor.

According to another embodiment of the present invention the method as defined above, additionally comprising step of selecting said vapor from a group consisting of anesthetic gas e.g., Nitrous oxide (N₂O), Halothane, Enflurane, Isoflurane, Sevoflurane, Desflurane and Xenon, water, oxygen or any combination thereof.

According to another embodiment of the present invention, the method additionally comprising the step of altering the vapor introduced into the chamber.

According to another embodiment of the present invention the method as defined above, additionally comprising step of extracting said anaesthesia from each of said chambers.

According to another embodiment of the present invention the method as defined above, additionally comprising step of ceasing said step of introducing said thermoregulated anaesthetic gas.

According to another embodiment of the present invention the method as defined above, additionally comprising step of controlling parameters selected from a group consisting of humidity, vapor concentration and any combination thereof in said chamber (100).

According to another embodiment of the present invention, the method additionally comprises step of ceasing the infusion of said thermoregulated anaesthetic gas into said chamber.

According to another embodiment of the present invention, the method additionally comprises step of placing at least one face-mask on said mammal; and delivering said vapor to said mammal at ambient temperature.

According to another embodiment of the present invention, the method additionally comprises step of sealing said system such that no leakage of said vapor is obtained.

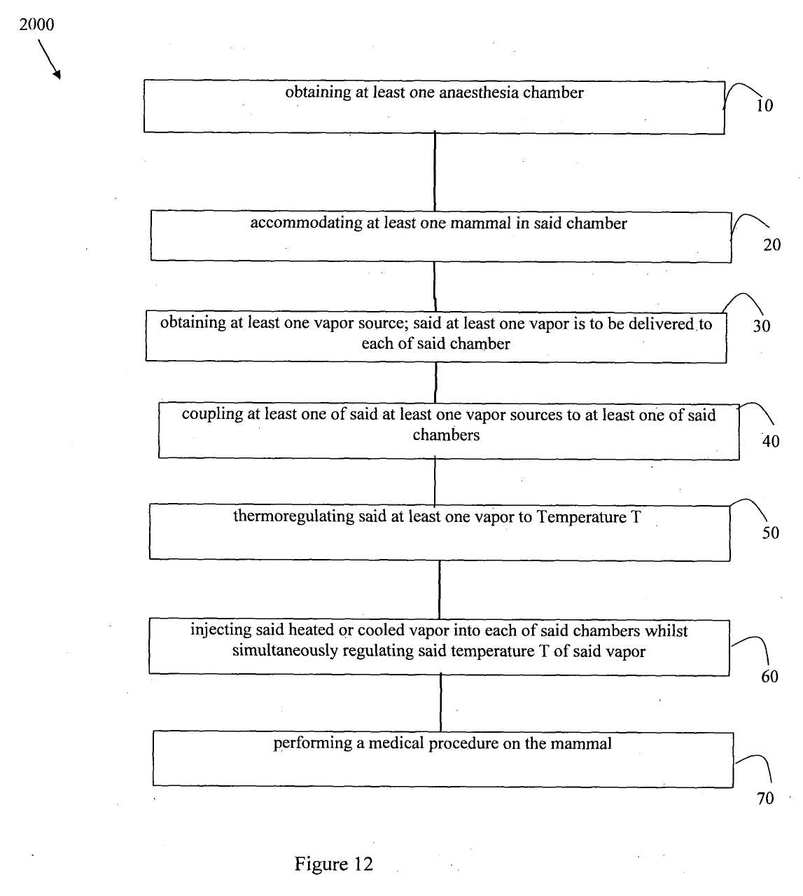

Reference is now made to FIG. 12, disclosing a method (2000) for performing at least one medical and/or biological procedure on at least one mammal. The method comprises steps selected inter alia from:
a. Obtaining at least one anaesthesia chamber (10);
b. Accommodating at least one mammal in the chamber (20);
c. Obtaining at least one vapor source which is to be delivered to each of the chambers (30);
d. Coupling at least one vapor sources to at least one of the chambers (40);
e. Thermoregulating the at least one vapor to Temperature T (50);
f. Introducing the thermoregulated vapor into each of the chambers whilst simultaneously regulating the temperature T of the vapor (60); and,
g. Performing a medical and/or biological procedure on the mammal (70).

According to another embodiment of the present invention, the method additionally comprises step of attuning said temperature T to be higher than about 0 and lower than about 100 degrees, especially 4 degrees C. to about 42 degrees C.

According to another embodiment of the present invention, the method additionally comprising the step of entirely filing the chamber with the vapor

According to another embodiment of the present invention, the method additionally comprising the step of at least partially purging the chamber from the vapor.

According to another embodiment of the present invention, the method additionally comprises step of selecting said vapor from a group consisting of anesthetic gas e.g., Nitrous oxide (N₂O), Halothane, Enflurane, Isoflurane, Sevoflurane, Desflurane and Xenon, water, oxygen or any combination thereof.

According to another embodiment of the present invention, the method additionally comprising the step of altering the vapor introduced into the chamber.

According to another embodiment of the present invention, the method additionally comprises step of extracting said anaesthesia from each of said chambers.

According to another embodiment of the present invention, the method additionally comprises step of ceasing said step of introducing said thermoregulated anaesthetic gas.

According to another embodiment of the present invention, the method additionally comprises step of controlling parameters selected from a group consisting of humidity, vapor concentration and any combination thereof in said chamber (100).

According to another embodiment of the present invention, the method additionally comprises step of placing at least one face-mask on said mammal; and delivering said vapor to said mammal at ambient temperature.

According to another embodiment of the present invention, the method additionally comprises step of performing another medical and/or biological procedure on said mammal.

According to another embodiment of the present invention, the method additionally comprises step of sealing said system such that no leakage of said vapor is obtained.

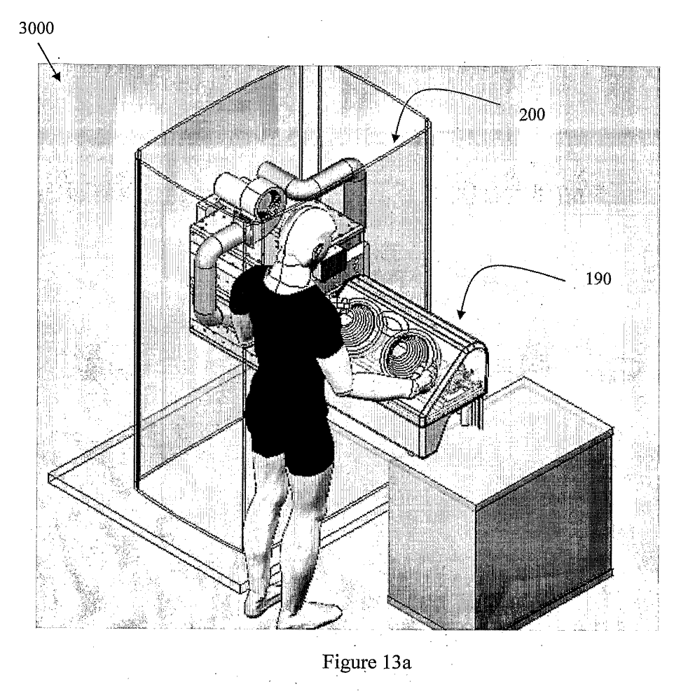

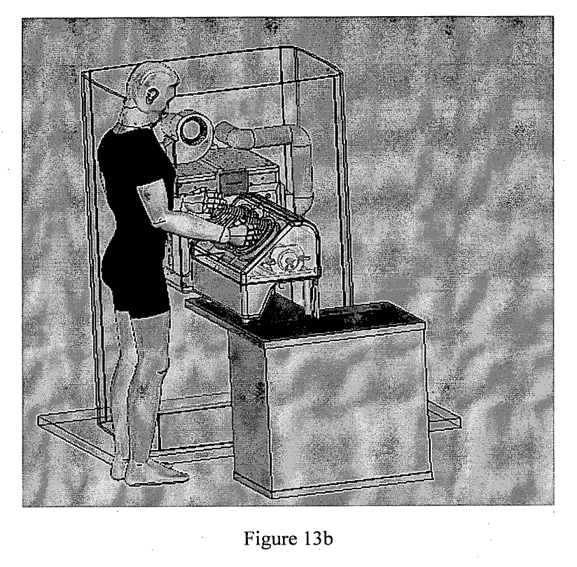

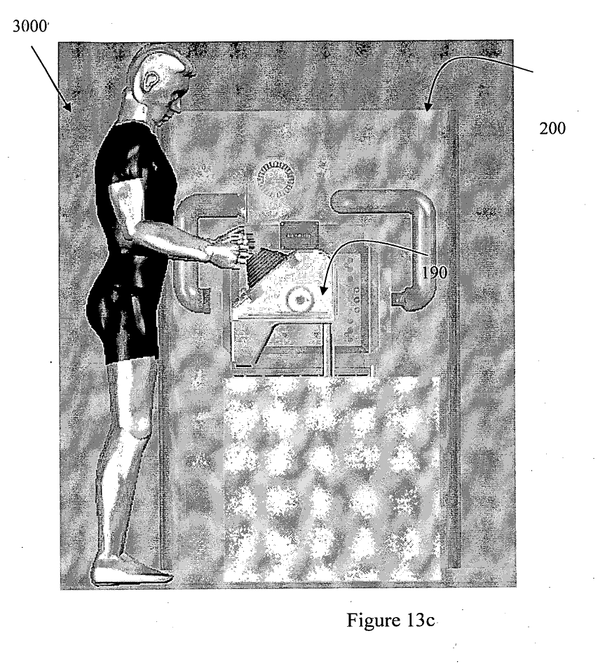

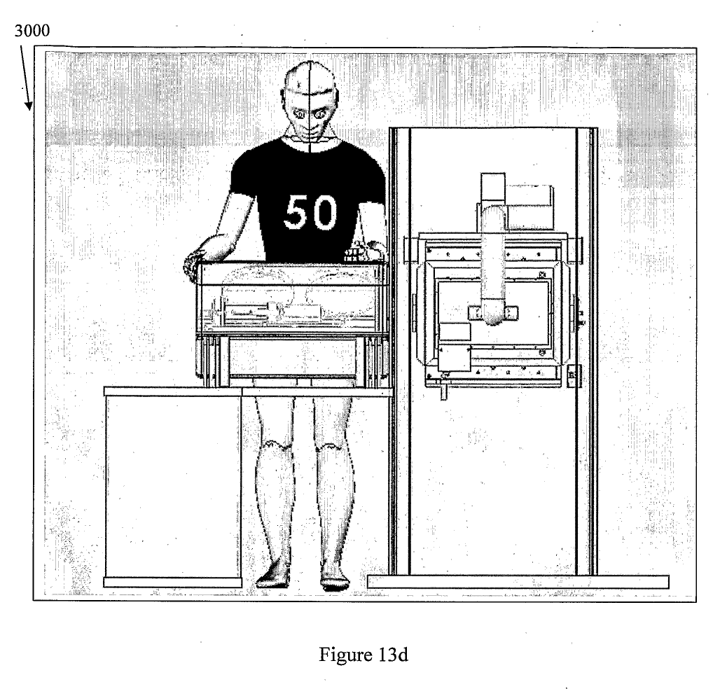

Reference is now made to FIGS. 13a-13d, schematically illustrating different views of another embodiment 3000 of the present invention. According to that embodiment, a glove box 190 is provided. The glove box 190 is adapted to accommodating the system 1000, as defined in any of the above. Said glove box 190 is adapted to enable any pretreatment to the mammal prior to performing any medical procedure on said mammal.

Additionally or alternatively, said embodiment 3000 may be in communication with treating mechanism 200 for performing said medical procedure. As mentioned above, the treating mechanism may an imaging device selected from a group consisting of NMR, MRI, CT, X-ray, ultrasound device, fluorescence device or thermographic device.

Embodiment 3000 enables any medical personal to pretreat the mammal prior to performing any medical and/or biological procedure.

According to said embodiment, treating the mammal will incorporate inter alia the following steps:
(a) obtaining at least one anaesthesia chamber and at least one glove box;
(b) accommodating said at least one chamber within said glove box. It is acknowledged that the chamber may or may not be sealed once introduced into the glove box.
(c) accommodating at least one mammal in said chamber;
(d) obtaining an anaesthetic gas sources; said anaesthetic gas is to be delivered to said chamber;
(e) coupling said anaesthetic gas sources to said chamber;
(f) thermoregulating said anaesthetic gas to Temperature T;
(g) injecting said thermoregulated anaesthetic gas into said chamber whilst simultaneously regulating said temperature T of said anaesthetic gas. It is emphasized that if the chamber was introduced into said glove box unsealed, than said glove box is filled with thermoregulated anaesthetic gas as well.
(h) pretreating said mammal accommodated within said chamber and said glove box;
(i) sealing said chamber. It should be pointed out that said thermoregulated anaesthetic gas may still be injected to said chamber;
(j) purging said thermoregulated anaesthetic gas from said glove box;
(k) extracting said chamber out of said glove box; and,
(l) introducing said chamber accommodating said mammal into any treating mechanism for performing any medical procedure on said mammal.

As mentioned above, the chamber, according to en embodiment, has a backing support (i.e., a bed) upon which the mammal is laid; and a top cover adapted to close and seal the chamber.

Furthermore, the chamber is coupled to the introducing (infusion) mechanism which are adapted to supply at least one thermoregulated vapor into the chamber.

It should also be appreciated that the above described description of methods and apparatus are to be interpreted as including apparatus for carrying out the methods, and methods of using the apparatus of any type as well known to a person or ordinary skill, and which need not be described in detail herein for enabling a person of ordinary skill to practice the invention.

For the main embodiments of the invention, the particular selection of type and model is not critical, though where specifically identified, this may be relevant. The present invention has been described using detailed descriptions of embodiments thereof that are provided by way of example and are not intended to limit the scope of the invention. No limitation, in general, or by way of words such as “may”, “should”, “preferably”, “must”, or other term denoting a degree of importance or motivation, should be considered as a limitation on the scope of the claims or their equivalents unless expressly present in such claim as a literal limitation on its scope. It should be understood that features and steps described with respect to one embodiment may be used with other embodiments and that not all embodiments of the invention have all of the features and/or steps shown in a particular figure or described with respect to one of the embodiments. That is, the disclosure should be considered complete from combinatorial point of view, with each embodiment of each element considered disclosed in conjunction with each other embodiment of each element (and indeed in various combinations of compatible implementations of variations in the same element). Variations of embodiments described will occur to persons of the art. Furthermore, the terms “comprise,” “include,” “have” and their conjugates, shall mean, when used in the claims, “including but not necessarily limited to.” Each element present in the claims in the singular shall mean one or more element as claimed, and when an option is provided for one or more of a group, it shall be interpreted to mean that the claim requires only one member selected from the various options, and shall not require one of each option. The abstract shall not be interpreted as limiting on the scope of the application or claims.

It is noted that some of the above described embodiments may describe the best mode contemplated by the inventors and therefore may include structure, acts or details of structures and acts that may not be essential to the invention and which are described as examples. Structure and acts described herein are replaceable by equivalents performing the same function, even if the structure or acts are different, as known in the art. Therefore, the scope of the invention is limited only by the elements and limitations as used in the claims.

1-97. (canceled)

98. A system (1000) for anaesthetizing mammals in a thermoregulated confined volume comprising:
a. at least one chamber (100) confining a volume in size and shape for accommodating at least one mammal;
b. supplying mechanism (120) for supplying at least one anesthetizing vapor to said at least one chamber (100);
c. temperature controlling mechanism (130), in communication with said vapor supplying mechanism (120), adapted to thermoregulate said vapor to temperature T; and,
d. vapor introducing mechanism (110), coupled to said supplying mechanism (120) and to said at least one chamber (100), adapted to introduce said thermoregulated vapor to said at least one chamber (100);
wherein said temperature T within said confined volume is regulated by said thermoregulated vapor.

99. The system according to claim 98, additionally comprising at least one element selected from a group consisting of (a) mechanism (150) for controlling parameters selected from a group consisting of humidity, vapor concentration and any combination thereof in said chamber (100); (b) selection mechanism coupled to said supplying mechanism (120), introducing mechanism (110) and said chamber (100) for selecting and/or altering between said vapor introduced into said chamber;
wherein said selection mechanism is adapted to select among a plurality of said vapors introduced into said chamber; (c) extracting mechanism (140) adapted to purge said thermoregulated vapor from said chamber (100); (d) at least one imaging device selected from a group consisting of NMR, MRI, CT, X-ray, ultrasound device, fluorescence device, thermography device and any combination thereof.

100. The system according to claim 98, wherein said chamber (100) comprising a backing support upon which said mammal lies; and a top cover adapted to close and/or seal said chamber.

101. The system according to claim 98, wherein at least a portion of said chamber (100) is disposable.

102. The system according to claim 98, wherein said temperature T is higher than about 0 degrees C. and lower than about 100 degrees C., especially 4 degrees C. to about 42 degrees C.

103. The system according to claim 98, wherein said anesthetizing vapor is selected from a group consisting of Nitrous oxide (N₂O), Halothane, Enflurane, Isoflurane, Sevoflurane, Desflurane and Xenon, water, oxygen any any combination thereof.

104. The system according to claim 98, wherein said temperature controlling mechanism (130) is a thermoregulator.

105. The system according to claim 98, adapted to anaesthetize simultaneously more than one mammal in the said chamber (100).

106. The system according to claim 98, wherein said system additionally comprising at least one face-mask to be placed on said mammal, adapted to deliver said vapor to said mammal at ambient temperature.

107. A system (1000) for simultaneously anaesthetizing a plurality of mammals in a thermoregulated confined volume, comprising:
a. a plurality of chambers (100), each of which confines a volume of size and shape capable of accommodating at least one mammal;
b. supplying mechanism (120) for supplying at least one anesthetizing vapor to each of said chambers (100);
c. temperature controlling mechanism (130), in communication with said vapor supplying mechanism (120), adapted to thermoregulate said vapor to temperature T; and,
d. a plurality of vapor introducing mechanisms (110), adapted to introduce said thermoregulated vapor to each of said chambers (100); each of said introducing mechanism coupled to at least one of said chambers (100);
wherein said temperature T within each of said confined volume is regulated by said thermoregulated vapor.

108. The system according to claim 107, wherein said chamber has a backing support upon which said mammal lies; and a top cover adapted to close and/or seal said chamber.

109. The system according to claim 107, additionally comprising at least one element selected from a group consisting of (a) a controlling mechanism (150) adapted to control parameters selected from the group consisting of humidity, vapor concentration and any combination thereof in each of said chambers (100); (b) extracting mechanism (140) adapted to purge said thermoregulated vapor from each of said chambers (100); and any combination thereof.

110. The system according to claim 107, wherein at least a portion of each of said chambers is disposable.

111. The system according to claim 107, wherein said temperature T is higher than about 0 degrees C. and lower than about 100 degrees, especially 4 degrees C. to about 42 degrees C.

112. The system according to claim 107, wherein said anesthetic vapor is selected from a group consisting of Nitrous oxide (N₂O), Halothane, Enflurane, Isoflurane, Sevoflurane, Desflurane and Xenon, water, oxygen and any combination thereof.

113. The system according to claim 107, additionally comprising selection mechanism coupled to said supplying mechanism, introducing mechanism and said chamber for selecting and/or altering between said vapor introduced into said chamber.

114. The system according to claim 107, wherein said temperature controlling mechanism (130) is selected from a group consisting of a thermoregulator, adapted to select among said vapors introduced into said chamber.

115. The system according to claim 107, wherein said system additionally comprising at least one face-mask to be placed on said mammal, adapted to deliver said vapor to said mammal at ambient temperature.

116. A system for performing a medical and/or biological procedure on mammals comprising:
a. at least one chamber (100) confining a volume in size and shape for accommodating at least one mammal;
b. supplying mechanism (120) for supplying at least one anesthetizing vapor to said at least one chamber (100);
c. temperature controlling mechanism (130), in communication with said vapor supplying mechanism (120), adapted to thermoregulate said vapor to temperature T;
d. at least one vapor introducing mechanism (110), each of which is coupled to at least one of said chambers; each of said introducing mechanism is adapted to introduce said thermoregulated vapor to each of said chambers (100); and,
e. treating mechanism adapted to perform said medical and/or biological procedure on said mammal;
wherein said temperature T within said confined volume is regulated by said thermoregulated vapor.

117. The system according to claim 116, wherein said chamber has a backing support upon which said mammal lies ; and a top cover adapted to close and/or seal said chamber.

118. The system according to claim 116, additionally comprising at least one element selected from a group consisting of (a) controlling mechanism (150) adapted to control parameters selected from a group consisting of humidity, vapor concentration and any combination thereof in said chamber (100); (b) selection mechanism coupled to said supplying mechanism, introducing mechanism and said chamber wherein said selection mechanism is adapted to select among said vapors introduced into said chamber; (c) selection mechanism coupled to said supplying mechanism, introducing mechanism and said chamber, wherein said selection mechanism is adapted to alter the vapors introduced into said chamber; (d) extracting mechanism (140) adapted to purge said thermoregulated vapor from said chamber (100); (e) at least one face-mask to be placed on said mammal, adapted to deliver said vapor to said mammal at ambient temperature; and any combination thereof.

119. The system according to claim 116, wherein at least a portion of said chamber is disposable.

120. The system according to claim 116, wherein said temperature T is higher than about 0 degrees C. and lower than about 100 degrees, especially 4 degrees C. to about 42 degrees C.

121. The system according to claim 116, wherein said anesthetic vapor is selected from a group consisting of Nitrous oxide (N₂O), Halothane, Enflurane, Isoflurane, Sevoflurane, Desflurane and Xenon, water, oxygen or any combination thereof.

122. The system according to claim 116, wherein said system is adapted to simultaneously anaesthetize more than one mammal in the said chamber.

123. The system according to claim 116, wherein said treating mechanism is at least one imaging device selected from a group consisting of NMR, MRI, CT, X-ray, ultrasound device, fluorescence device, thermography device or any combination thereof.

124. A method for anaesthetizing mammals in a thermoregulated confined volume, wherein said method comprising:
a. obtaining an anesthesia chamber;
b. accommodating at least one mammal in said chamber;
c. obtaining an anaesthetic vapor sources adapted to deliver anaesthetic vapor to each of said chamber;
d. coupling said anaesthetic vapor sources to said chamber;
e. thermoregulating said anaesthetic vapor to Temperature T; and,
f. introducing said thermoregulated anaesthetic vapor into said chamber thereby regulating said temperature T within said chamber.

125. The method according to claim 124, additionally comprising step of medically treating said mammal.

126. The method according to claim 125, wherein said step of treating said mammal additionally comprising at least one step selected from (a) performing any medical and/or biological procedure on said mammal; (b) introducing said chamber accommodating said mammal into treating mechanism; and any combination thereof.

127. The method according to claim 124, additionally comprising step of attuning said temperature T to be higher than about 0 degrees C. and lower than about 100 degrees, especially 4 degrees C. to about 42 degrees C.

128. The method according to claim 124, additionally comprising step of selecting said anesthetic vapor from a group consisting of Nitrous oxide (N₂O), Halothane, Enflurane, Isoflurane, Sevoflurane, Desflurane and Xenon, water, oxygen or any combination thereof.

129. The method according to claim 124, additionally comprising at least one step selected from (a) altering said vapor introduced into said chamber; (b) entirely filing said chamber with said vapor; (c) at least partially purging said vapor from said chamber; (d) ceasing said step of introducing said thermoregulated anaesthetic vapor; and any combination thereof.

130. The method according to claim 124, additionally comprising step of controlling parameters selected from a group consisting of humidity, vapor concentration and any combination thereof in said chamber (100).

131. The method according to claim 124, additionally comprising step of placing at least one face-mask on said mammal; and delivering said vapor to said mammal at ambient temperature.

132. The method according to claim 124, additionally comprising step of sealing said system such that no leakage of said vapor is obtained.

133. A method for simultaneously anaesthetizing a plurality of mammals in a thermoregulated confined volume, said method comprising:
a. obtaining a plurality of anaesthesia chambers;
b. accommodating at least one mammal in each of said chambers;
c. obtaining at least one an anaesthetic vapor source, adapted to deliver; said anaesthetic vapor to each side of each of said chambers;
d. coupling said anaesthetic vapor source to said plurality of chambers;
e. thermoregulating said anaesthetic vapor to Temperature T; and,
f. introducing said thermoregulated anaesthetic vapor into each of said chambers thereby regulating said temperature T within said chamber.

134. The method according to claim 133, additionally comprising step of treating said mammal.

135. The method according to claim 134, wherein said step of treating said mammal additionally comprising at least one step selected from (a) performing any medical and/or biological procedure on said mammal; (b) introducing said chamber accommodating said mammal into treating mechanism; and any combination thereof.

136. The method according to claim 133, additionally comprising at least one step selected from (a) attuning said temperature T to be higher than about 0 degrees C. and lower than about 100 degrees, especially 4 degrees C. to about 42 degrees C.; (b) selecting said anesthetic vapor from a group consisting of Nitrous oxide (N₂O), Halothane, Enflurane, Isoflurane, Sevoflurane, Desflurane and Xenon, water, oxygen or any combination thereof; (c) altering said vapor introduced into said chamber; (d) entirely filling said chamber with said vapor; (e) at least partially purging said chamber from said vapor; (f) ceasing said step of introducing said thermoregulated anaesthetic vapor; and any combination thereof.

137. The method according to claim 133, additionally comprising step of controlling parameters selected from a group consisting of humidity, vapor concentration and any combination thereof in said chamber (100).

138. The method according to claim 133, additionally comprising step of placing at least one face-mask on said mammal; and delivering said vapor to said mammal at ambient temperature.

139. The method according to claim 133, additionally comprising step of sealing said system such that no leakage of said vapor is obtained.

140. A method for simultaneously anaesthetizing a plurality of mammals in a thermoregulated confined volume, said method comprising:
a. obtaining a plurality of anaesthesia chambers;
b. accommodating at least one mammal in each of said chamber;
c. obtaining a plurality of anaesthetic vapor sources; said anaesthetic vapor is to be delivered to each of said chamber;
d. coupling each of said anaesthetic vapor sources to at least one of said chambers;
e. thermoregulating said anaesthetic vapor to Temperature T; and,
f. introducing said thermoregulated anaesthetic vapor into each of said chambers thereby regulating said temperature T within said chamber.

141. The method according to claim 140, additionally comprising step of treating said mammal.

142. The method according to claim 141, wherein said step of treating said mammal additionally comprising at least one step selected from (a) of performing any medical and/or biological procedure on said mammal; (b) introducing said chamber accommodating said mammal into treating mechanism; or any combination thereof.

143. The method according to claim 140, additionally comprising step of attuning said temperature T to be higher than about 0 and lower than about 100 degrees, especially 4 degrees C. to about 42 degrees C.

144. The method according to claim 140, additionally comprising step of selecting said anesthetic vapor from a group consisting of Nitrous oxide (N₂O), Halothane, Enflurane, Isoflurane, Sevoflurane, Desflurane and Xenon, water, oxygen or any combination thereof.

145. The method according to claim 140, additionally comprising step of additionally comprising a selection mechanism coupled to said supplying mechanism, introducing mechanism and said chamber, wherein said selection mechanism is adapted for altering said vapor introduced into said chamber.

146. The method according to claim 140, additionally comprising at least one step selected from (a) entirely filing said chamber with said vapor; (b) at least partially purging said chamber from said vapor; (c) ceasing said step of introducing said thermoregulated anaesthetic vapor; (d) placing at least one face-mask on said mammal; and delivering said vapor to said mammal at ambient temperature; and any combination thereof.

147. The method according to claim 140, additionally comprising step of controlling parameters selected from a group consisting of humidity, vapor concentration and any combination thereof in said chamber (100).

148. The method according to claim 140, additionally comprising step of sealing said system such that no leakage of said vapor is obtained.

149. A method for performing at least one medical and/or biological procedure on at least one mammal in a thermoregulated confined volume, said method comprising:
a. obtaining at least one anaesthesia chamber;
b. accommodating at least one mammal in said chamber;
c. obtaining at least one anesthetizing vapor source; said at least one vapor is to be delivered to each of said chamber;
d. coupling at least one of said at least one vapor sources to at least one of said chambers;
e. thermoregulating said at least one vapor to Temperature T;
f. introducing said thermoregulated vapor into each of said chambers thereby regulating said temperature T within said chamber; and,
g. performing said medical and/or biological procedure on said at least one mammal.

150. The method according to claim 149, wherein said step of treating said mammal additionally comprising step of introducing said chamber accommodating said mammal into treating mechanism.

151. The method according to claim 150, additionally comprising step of attuning said temperature T to be higher than about 0 and lower than about 100 degrees, especially 4 degrees C. to about 42 degrees C.

152. The method according to claim 149, additionally comprising step of selecting said anesthetic vapor from a group consisting of Nitrous oxide (N₂O), Halothane, Enflurane, Isoflurane, Sevoflurane, Desflurane and Xenon, water, oxygen or any combination thereof.

153. The method according to claim 149, additionally comprising at least one step selected from (a) altering said vapor introduced into said chamber; (b) entirely filing said chamber with said vapor; (c) at least partially purging said chamber from said vapor; (d) ceasing said step of introducing said thermoregulated anaesthetic vapor; and any combination thereof.

154. The method according to claim 149, additionally comprising step of controlling parameters selected from a group consisting of humidity, vapor concentration and any combination thereof in said chamber (100).

155. The method according to claim 149, additionally comprising step of placing at least one face-mask on said mammal; and delivering said vapor to said mammal at ambient temperature.

156. The method according to claim 149, additionally comprising step of performing another medical and/or biological procedure on said mammal.

157. The method according to claim 149, additionally comprising step of sealing said system such that no leakage of said vapor is obtained.

158. A system for performing a medical and/or biological procedure on mammals, comprising:
a. at least one chamber (100) confining a volume in size and shape for accommodating at least one mammal;
b. at least one pretreatment mechanism adapted to accommodate said chamber (100) and to enable pretreatment of said mammal;
c. supplying mechanism (120) for supplying at least one anesthetizing vapor to said at least one chamber (100);
d. temperature controlling mechanism (130), in communication with said vapor supplying mechanism (120), adapted to thermoregulate said vapor to temperature T;
e. at least one vapor introducing mechanism (110), each of which is coupled to at least one of said chambers; each of said introducing mechanism is adapted to introduce said thermoregulated vapor to each of said chambers (100);
f. treating mechanism adapted to perform said medical and/or biological procedure on said mammal;
wherein said temperature T within said confined volume is regulated by said thermoregulated vapor.

159. The system according to claim 158, wherein said pretreatment mechanism are selected form a group consisting of glove box.

160. The system according to claim 158, wherein said chamber has a backing support upon which said mammal lays; and a top cover adapted to close and/or seal said chamber.

161. The system according to claim 158, additionally comprising at least one element selected from (a) controlling mechanism (150) adapted to control parameters selected from a group consisting of humidity, vapor concentration and any combination thereof in said chamber (100); (b) selection mechanism coupled to said supplying mechanism, introducing mechanism and said chamber for selecting and/or altering between said vapor introduced into said chamber; (c) extracting mechanism (140) adapted to purge said thermoregulated vapor from said chamber (100); (d) at least one face-mask to be placed on said mammal, adapted to deliver said vapor to said mammal at ambient temperature; and any combination thereof.

162. The system according to claim 158, wherein at least a portion of said chamber is disposable.

163. The system according to claim 158, wherein said temperature T is higher than about 0 and lower than about 100 degrees, especially 4 degrees C. to about 42 degrees C.

164. The system according to claim 158, wherein said anesthetic vapor is selected from a group consisting of Nitrous oxide (N₂O), Halothane, Enflurane, Isoflurane, Sevoflurane, Desflurane and Xenon, water, oxygen or any combination thereof.

165. The system according to claim 158, wherein said temperature controlling mechanism (130) is selected from a group consisting of a thermoregulator.

166. The system according to claim 158, wherein said treating mechanism is at least one imaging device selected from a group consisting of NMR, MRI, CT, X-ray, ultrasound device, fluorescence device, thermographic device or any combination thereof.
